(19) United States
(12) Patent Application Publication
(10) Pub. No.: US 2026/0297128 A1
(43) Pub. Date: Oct. 1, 2026

(54) PEPTOID COMPOSITIONS TARGETING RAS PROTEINS AND METHODS OF TREATMENT OF CANCER USING THE SAME

(71) Applicant: University of Houston System, Houston, TX (US)

(72) Inventors: Damith Gomika UDUGAMASOORIYA, Houston, TX (US); Eunsun PARK, Houston, TX (US)

(21) Appl. No.: 19/479,530

(22) PCT Filed: May 8, 2024
(86) PCT No.: PCT/US2024/028430

§ 371 Date: Oct. 28, 2025

Related U.S. Application Data
(60) Provisional application No. 63/464,920, filed on May 8, 2023.

(51) Int. Cl. C07K 7/06; A61K 31/4439; A61K 31/519; A61K 38/00; A61P 35/00

(52) U.S. Cl. CPC C07K 7/06; A61K 31/4439; A61K 31/519; A61P 35/00; A61K 38/00

(57) ABSTRACT

Peptoid compositions targeting RAS proteins and methods of treatment of cancers using these peptoid compositions are described here. Also provided are fatty acid conjugated peptoid compositions that interact with KRAS, HRAS, or NRAS proteins. Certain peptoid compositions inhibit function of a KRAS protein in a cancer cell. The KRAS protein can be a mutant or wild type KRAS protein. The peptoid composition can be administered along with a MEK inhibitor or an ERK inhibitor.

### TECHNICAL FIELD

The disclosure relates to peptoids that interact with a RAS protein and methods of treatment of cancers using the peptoid compositions.

### BACKGROUND

Cancer is one of the leading causes of deaths worldwide, and the total annual cancer deaths have continued to rise over time. Cancer therapies vary by cancer types, stages, and even individual patients and include one or more of the following: surgery, chemotherapy, targeted therapy, hormone therapy, and immunotherapy. The conventional drugs target various cancer related protein biomarkers. These protein biomarkers vary from cancer types, stages, and even individual patients and this heterogeneity restricts developing the universal cure for cancer. A single drug that can be used to treat multiple cancer types will significantly impact the clinical treatment system, as current drugs are restricted for a particular cancer type or sub type or even for some patients. This is economically untenable. Due to the complexity of cancers, currently the clinicians have to personalized medications with a huge cost, still with unexpected failures.

RAS proteins are small guanosine triphosphatases that regulate cell growth, differentiation, and apoptosis, and include the Kirsten rat sarcoma viral oncogene homologue (KRAS), Harvey rat sarcoma viral oncogene homolog (HRAS), or the Neuroblastoma rat sarcoma viral oncogene homolog (NRAS). KRAS is one of the best-known key oncogenes in cancers with high mutation rates and is commonly expressed in many cancer types. KRAS protein is one of the most common oncogenes that mutates in roughly one-third of all cancers. The KRAS protein is a member of a superfamily of guanosine-5-triphosphatase proteins. However, some characteristics, including (i) difficulty to develop conventional competitive inhibitors for GDP and GTP due to the high binding affinity with picomolar level, (ii) lack of deep and well-defined surface pockets, and (iii) conserved residues except one residue, make it one of the most hard-to-hit targets in cancer research. Thus, KRAS has been dubbed as “undruggable” over forty years.

Only two small molecular drugs have been developed recently directly targeting mutant KRAS G12C, while targeting other KRAS-related proteins indirectly. Those two small molecules covalently bind to mutant KRAS G12C and are not effective on other KRAS and RAS mutation types. Antibodies have poor cell permeability. Linear peptides cannot adequately cover the large protein-protein interaction surface (PPI) of KRAS and they may also have challenges related to cell membrane permeability and low biostability. The small molecular drugs need well-defined and deep binding pockets on targeted proteins, but KRAS has shallow and large surface. Therefore, the conventional drugs such as small molecules, antibodies, and peptides have restraint as KRAS inhibitors. In addition, recent studies demonstrated that presence of non-mutant WT KRAS develop resistance of downstream signaling inhibitors in KRAS-amplified or mutant KRAS cancers.

### SUMMARY

Provided here are compositions and methods to address these shortcomings of the art and provide other additional or alternative advantages. The disclosure herein provides one or more peptoid compositions that interact with KRAS, HRAS, or NRAS proteins and have efficacy in wider range of patients having different cancer types. Embodiments of the disclosure address major unmet needs in oncology drug discovery research and in the clinic. Certain embodiments of the peptoids include a minimum pharmacophore provided as general formula I or a pharmaceutically acceptable salt thereof. Certain embodiments of the peptoids include a peptoid having the structure of general formula II or III or a pharmaceutically acceptable salt thereof. Certain embodiments include a peptoid with a C₄-C₁₈ fatty acid at a C-terminal of the structure of general formula I or II or III or a pharmaceutically acceptable salt thereof. The fatty acid can be myristic acid, palmitic acid, or stearic acid. The fatty acid can be an unsaturated fatty acid, palmitoleic acid, or oleic acid. Certain embodiments of the peptoids include a fatty acid conjugated peptoid having the structure of general formula II or III or a pharmaceutically acceptable salt thereof. Certain embodiments of the peptoids include a fatty acid conjugated peptoid having the structure of general formula IV or V or VI or a pharmaceutically acceptable salt thereof. Certain embodiments of the fatty acid conjugated peptoid include a linker. In certain embodiments, the linker can be one or more amino acids. In certain embodiments, the linker is up to five amino acids long. In certain embodiments, the fatty acid is coupled with ε-amino group of lysine at the C-terminal of the peptoid. Therefore the linker in this embodiment is one lysine residue.

Embodiments include methods for treating cancer by administering to a subject suffering from or susceptible to cancer a pharmaceutical composition containing a peptoid inhibitor of a function of KRAS, HRAS, or NRAS. In certain embodiments, the peptoid inhibitor is a pan-KRAS inhibitor that does not discriminate between KRAS mutants. In certain embodiments, the cancer cell has an overexpression of wild type KRAS protein as compared to a normal cell. In certain embodiments, the KRAS is a mutant KRAS, which can be a KRASG12V, KRASG12C, KRASG12D, or KRASQ61H protein. In certain embodiments, the KRAS is a wild-type KRAS. The peptoid inhibitors can be one or more peptoids corresponding to general formula II or III or a pharmaceutically acceptable salt thereof. The pharmaceutical composition can contain a fatty acid conjugated peptoid having the structure of general formula IV or a pharmaceutically acceptable salt thereof. The peptoid inhibitors can be a myristic acid conjugated peptoid corresponding to general formula V, a palmitic acid conjugated peptoid corresponding to general formula VI, and stearic acid conjugated peptoid corresponding to general formula VII. The cancer can be an adenocarcinoma. The cancer can be pancreatic c adenocarcinoma, lung adenocarcinoma, colon adenocarcinoma, colorectal adenocarcinoma, or rectal adenocarcinoma.

In certain embodiments, the method further includes administering an inhibitor of the mitogen-activated extracellular signal-regulated kinase (MEK) along with the pharmaceutical composition containing the peptoid as a combination therapy to the subject suffering from cancer. In certain embodiments, the method further includes administering an inhibitor of extracellular signal-regulated kinase (ERK) along with the pharmaceutical composition containing the peptoid as a combination therapy to the subject suffering from cancer. For example, the combined administration of a MEK inhibitor or an ERK inhibitor along with the peptoid composition can be especially useful for treatment of cancers that are resistant to MEK and ERK inhibitors. The MEK inhibitor can be trametinib. The ERK inhibitor can be ulixertinib.

Numerous other aspects, features and benefits of the present disclosure may be made apparent from the following detailed description taken together with the drawings. The pharmaceutical compositions can include compounds described herein, other components, or ingredients depending on desired prevention and treatment goals. It should be further understood that both the foregoing general description and the following detailed description are exemplary and illustrative and are not intended to limit the scope of any claimed embodiment.

### BRIEF DESCRIPTION OF THE DRAWINGS

The accompanying drawings, which are included to provide a further understanding of the embodiments of the present disclosure, are incorporated in and constitute a part of this specification, illustrate embodiments of the present disclosure, and together with the detailed description, serve to explain principles of the embodiments discussed herein. No attempt is made to show structural details of this disclosure in more detail than may be necessary for a fundamental understanding of the embodiments discussed herein and the various ways in which they may be practiced.

FIG. 1 is a diagrammatic representation of the novel approach disclosed herein showing a 3D-peptoid targeting KRAS and covering a large protein-protein interaction (PPI) surface on KRAS.

FIG. 2A is a schematic representation of biotinylated 3D-peptoid bound his-tagged KRAS identification via streptavidin-Qdots 655. FIG. 2B is a photograph of the isolated ‘hit l’ bead after a primary screen and a secondary screen using procedure illustrated in FIG. 2A. FIG. 2C is a photograph of the isolated ‘hit 2’ bead after a primary screen and a secondary screen using procedure illustrated in FIG. 2A.

FIG. 3A presents the chemical structure of biotinylated parent PKR1-B, PKR1.Met-B with Methionine, PKR1.Ala-B with Alanine, monomeric PKR1.M-B and scrambled PKR1.S-B utilized in an ELISA-like binding assay.

FIG. 3B and FIG. 3C are graphical representations of the concentration dependent WT KRAS binding curve of PKR1 derivatives, PKR1.M, and PKR1.S in GDP and GPPNHP solutions, respectively. FIG. 3D and FIG. 3E are graphical representations of the concentration dependent KRAS G12V binding curve of PKR1 derivatives, PKR1.M, and PKR1.S in GDP and GPPNHP solutions, respectively. FIG. 3F presents the Kd value of PKR1, PKR1.Met, PKR1.Ala, PKR1.S, and PKR1.M in WT KRAS and KRAS G12V with GDP and GPPNHP solutions.

FIG. 4A is a graphical representation of the sarcosine derivatives binding results of PKR1 to KRASG12V. FIG. 4B provides a mapping of the results from the pharmacophore studies mapped onto the structure of PKR1.

FIGS. 5A-5F are graphical representations of the cell viability results of PKR1 and PKR1.Met in (A) H1993, (B) H1299, (C) H1693, (D) H441, (E) Capan2, (F) H358, and (G) H358 cell lines.

FIGS. 6A-6C are graphical representations of the effects of the fatty acid conjugated PKR1 derivatives on cell survival and proliferation of (A) H441, (B) H358, and (C) H1993 cell lines.

FIG. 6D presents the IC50 values of fatty acid conjugated PKR1 derivatives in H441, H1993, and H358 cell lines.

FIG. 7A is an illustration of the scheme of timeline MTS assay. FIGS. 7B-7E are graphical representations of the cell viability following treatment with PKR1 and PKR1-C16 in H441 cell lines with (B) one time treatment, (C) two-time treatment at 0 D and 1 D, (D) two-time treatment at 0 D and 2 D, and (E) daily treatment. FIGS. 7F-7I are graphical representations of the cell viability following treatment with PKR1 and PKR1-C16 in H1993 cell lines with (F) one time treatment, (G) two-time treatment at 0 D and 1 D, (H) two-time treatment at 0 D and 2 D, and (I) daily treatment.

FIG. 8A is a set of representative images of colonies for H441 after 9 D incubation with a concentration gradient of PKR1-C16. Black dots represent colonies. FIG. 8B is a graphical representation presenting the dose-response relationship of number of colonies in H441 cells.

FIG. 9A is a set of representative images of wound healing for H441 cell lines after 2 D incubation with a concentration gradient of PKR1-C16. Magnification 20×. FIG. 9B is a graphical representation presenting the dose-response relationship of cell-free area in H441 cells.

FIGS. 10A-10I are graphical representations of the cell viability results of PKR1 derivatives in (A) HBECK3KT, (B) H1693, (C) H1993, (D) H1299, (E) H1395, (F) H441, (G) H358, (H) H2122, and (I) H1155 cell lines.

FIG. 11A is an illustration presenting that heterozygous KRAS mutant cells are resistant to the inhibition of KRAS downstream signaling while homozygous KRAS mutant cells are sensitive to such inhibition, therefore blocking wild type KRAS potentially re-sensitizes the heterozygous KRAS mutant cells to the inhibition of KRAS downstream signaling. FIG. 11B is a graphical representation presenting the cell viability of H441 harboring heterozygous KRAS G12V with co-treatments of Trametinib (upper panel) and Ulixertinib with PKR1-C16 (lower panel).

FIGS. 12A-12F are TIRF microscopy images of (A) DiO, (B) AF647-PKR1-C16, and (C) merge of DiO and AF647-PKR1-C16, and confocal microscopy images of (D) DiO, (E) AF647-PKR1-C16, and (F) merge of DiO and AF647-PKR1-C16.

FIG. 13 presents a schematic strategy to identify the most potential binding mode of PKR1 derivatives on KRAS.

FIGS. 14A and 14B present molecular docking poses of PKR1 to (A) GDP bound KRAS and (B) GMPPNP bound KRAS.

FIG. 15 presents the molecular docking results of PKR1-C14 to GMPPNP bound KRAS.

FIG. 16 presents the overview of the mechanism of 3D-peptoid for KRAS signaling and schematic representation of membrane anchored 3D-peptoid structure.

### DETAILED DESCRIPTION

Provided herein are pan-KRAS pharmaceutical compositions containing a 3-dimensional (3D) peptoid. Unlike the linear peptides, the 3D peptoids have multi-site binding chemical moieties that are spread over a large surface area (~1300-1500 Å²) on KRAS. These multi-hotspot-targeted interactions will lead to: (i) stronger binding, (ii) affecting the conformational changes needed for the activation of KRAS, as well as (iii) disrupting protein-protein interaction (PPI) with its upstream and downstream signaling proteins.

The conventional drug classes such as small molecules, antibodies, and peptides as KRAS inhibitors have failed due to multiple reasons over 4 decades. The small molecular drugs cannot effectively bind to shallow and large protein-protein interaction (PPI) surface area of KRAS. Antibodies and peptides may bind but are limited by cell membrane permeability issues. Furthermore, even linear peptides or peptidomimetics cannot adequately cover the large PPI surface (~1300-1500 Å²) of KRAS. The small molecular drugs need well-defined and deep binding pockets on targeted proteins, but KRAS has shallow and large surface. Antibodies may bind but not penetrate the cell membrane. Linear peptides cannot adequately cover the large protein-protein interaction surface (PPI) of KRAS and also can have cell membrane permeability issue as well as low biostability. The 3D-peptoids disclosed herein cover the shallow and large PPI surface area on KRAS to enhance the binding recognition and then interrupt PPI with the upstream and downstream signaling proteins. These peptoids are highly biologically amenable with high serum stability, tissue permeability, and are also non-immunogenic. These peptoids are also stable with higher shelf-life, easier and economical to develop as medications than current clinically applied drug classes such as small molecules, peptides and antibodies.

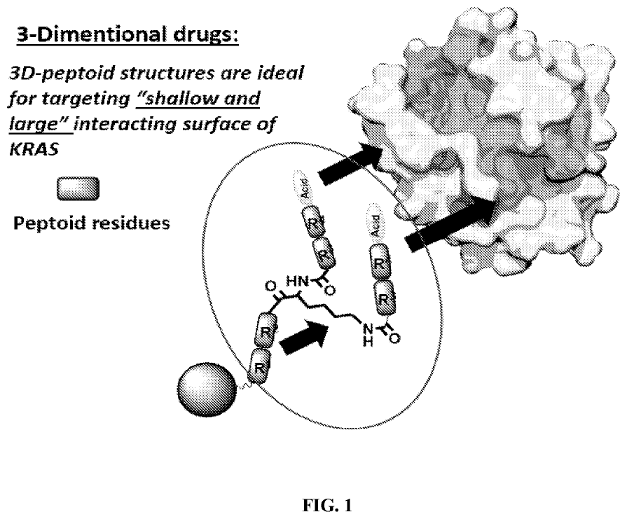

The 3D peptoids have multi-site binding chemical moieties that are spread over a large surface area (~1300-1500 Å²) on KRAS (FIG. 1). FIG. 1 is a diagrammatic representation of the novel approach disclosed herein showing a 3D-peptoid targeting KRAS and covering a large protein-protein interaction (PPI) surface on KRAS.

These multi-hotspot-targeted interactions can lead to stronger binding, affecting the conformational changes needed for the activation of KRAS, as well as disrupting PPIs with its upstream and downstream signaling proteins. In addition, peptoids have the improved cell permeability and resistance to degradation by proteases compared to the conventional peptide molecules. Hence, these properties of the 3D-peptoids render “undruggable” KRAS proteins into “druggable” targets. In addition, fatty acid conjugation enables these peptoids to anchor onto the cell membrane enhancing their binding to membrane-anchored targets, and concurrently improves the cell membrane permeability of the peptoids. Consequently, the fatty acid conjugation can enhance the cellular activity of the 3D-peptoid compositions.

Embodiments include peptoid compositions that interact with KRAS, HRAS, or NRAS proteins. In certain embodiments, the KRAS protein is a mutant KRAS, which can be one of a KRASG12V, KRASG12C, KRASG12D, or KRASQ61H protein. Embodiments include peptoid compositions that interact with mutant KRAS proteins and have efficacy in wider range of patients having different cancer types.

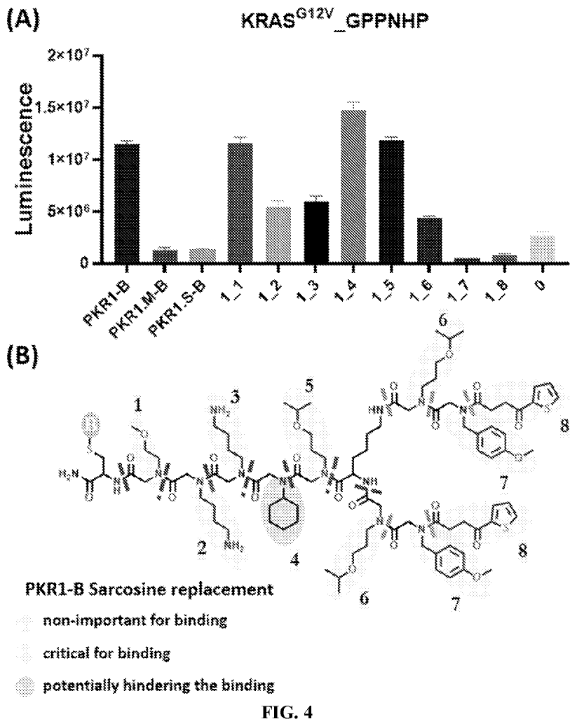

Certain embodiments of the peptoids include one possible minimum pharmacophore of PKR1 provided as general formula I or a pharmaceutically acceptable salt thereof. For example, each of the moieties labeled 1-8 of General Formula I as shown in FIG. 4 are subject to optimization with respect to serum stability, cell permeability, and non-immunogenicity, among other characteristics. Embodiments of the pan-KRAS pharmaceutical compositions contain a 3D-peptoid that has an initial linear range built with two variable peptoid units (R1 and R2) and branched regions with each branch consisting of at least two variable peptoid units (R3 and R4), and an organic moiety. In certain embodiments, the organic moiety contains an organic acid. In certain embodiments, the organic moiety can be one or more of the organic acids described below as a R5 group. As indicated in arrows in Formula 1 below, all these different chemical moieties can identify individual hot spots spread on the large PPI surface of the KRAS. The theoretical diversity of the library is about 144,500

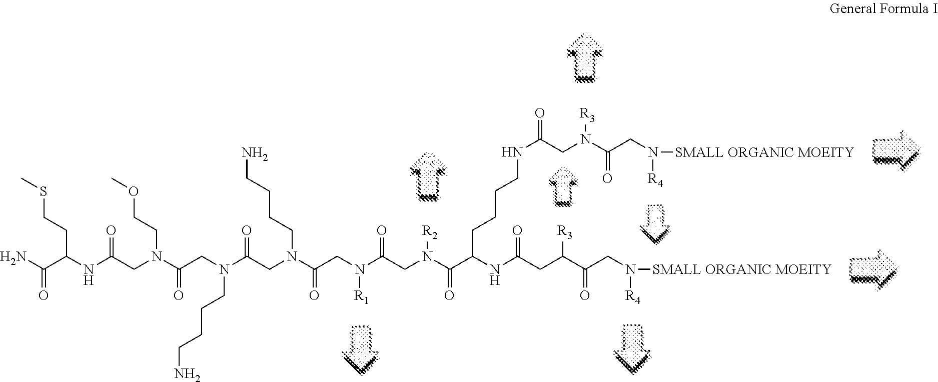

Embodiments include peptoid compositions of General Formula I that interact with KRAS, HRAS, or NRAS proteins. In certain embodiments of the peptoids, the R1, R2, R3, and R4 groups and the organic moiety (R5) are one or more of the following:
R1: 3-methylbutan-1-amine, Butan-2-amine, Propan-2-amine, 2-Methoxyethan-1-amine, 3-isopropoxypropan-1-amine, 3-Methoxypropan-1-amine, Butane-1,4-diamine, Furan-2-ylmethanamine
R2: 3-Methylbutan-1-amine, Butan-2-amine, Propan-2-amine, 2-Methoxyethan-1-amine, 3-isopropoxypropan-1-amine, Prop-2-yn-1-amine
R3: 3-methylbutan-1-amine, Butan-2-amine, Propan-2-amine, 2-Methoxyethan-1-amine, 3-isopropoxypropan-1-amine, 3-Methoxypropan-1-amine, Butane-1,4-diamine, Furan-2-ylmethanamine
R4: 3-methylbutan-1-amine, Butan-2-amine, Propan-2-amine, 2-Methoxyethan-1-amine, 3-isopropoxypropan-1-amine, 3-Methoxypropan-1-amine, Butane-1,4-diamine, Furan-2-ylmethanamine, Prop-2-yn-1-amine, (4-Chlorophenyl) methanamine, (4-Methoxyphenyl) methanamine, Pyridin-2-ylmethanamine
R5: 4-Oxo-4-(thiophen-2-yl)butanoic acid, 2-(3-Oxo-3,4-dihydro-2H-benzo[b][1,4]thiazin-2-yl)acetic acid, 2-(4-Chloro-2-methylphenoxy) acetic acid, 2-(4-Oxo-2-thioxothiazolidin-3-yl)acetic acid, 3-(5-Phenyl-1,3,4-oxadiazol-2-yl) propanoic acid

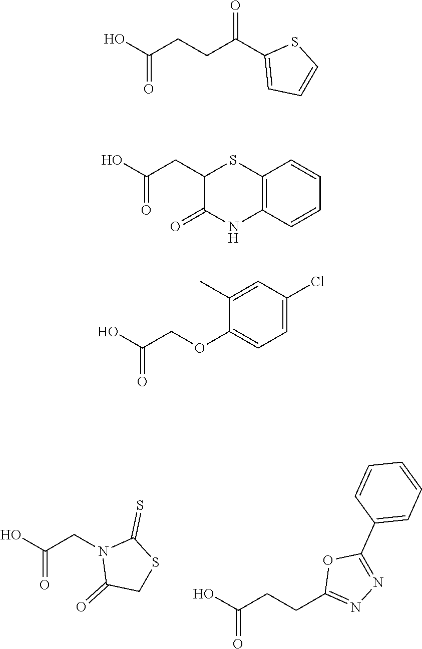 [SMILES: O=C(O)CCC(=O)c1cccs1 ; O=C(O)CC1Sc2ccccc2NC1=O ; Cc1cc(Cl)ccc1OCC(=O)O ; O=C(O)CN1C(=O)CSC1=S ; O=C(O)CCc1nnc(-c2ccccc2)o1]

Certain embodiments of the peptoids that interact with KRAS, HRAS, or NRAS proteins include a peptoid having the structure of general formula II or III or a pharmaceutically acceptable salt thereof. The pan-KRAS inhibitor function of these two illustrative peptoid compounds, PKR1 and PKR2, have been evaluated herein.

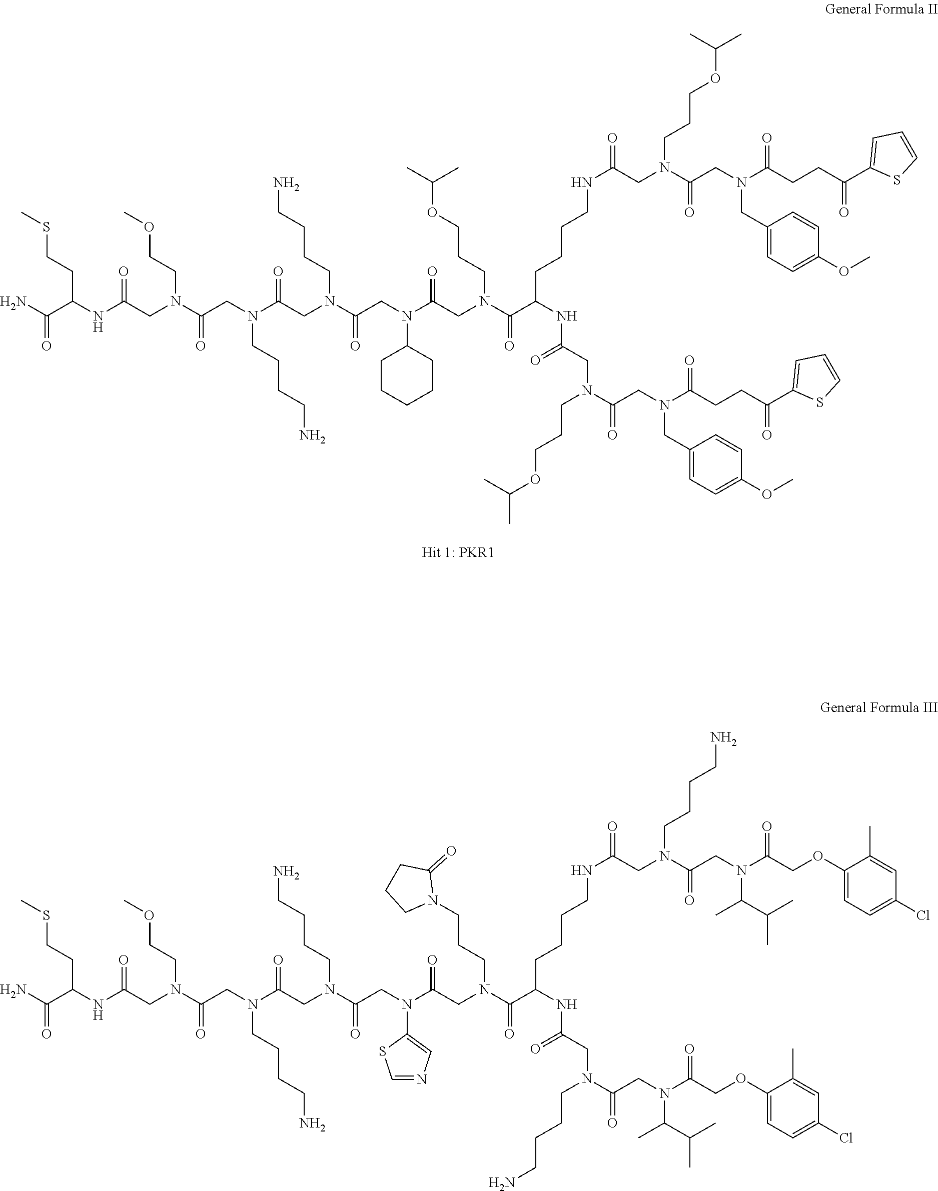

Certain embodiments of the peptoids include a fatty acid conjugated peptoid having the structure of general formula II or III or a pharmaceutically acceptable salt thereof. These fatty acids can be one or more of C₄-C₁₈ straight chain saturated fatty acid. In some embodiments, the fatty acid is an unsaturated fatty acid such as palmitoleic acid and oleic acid, a monounsaturated fatty acid, or a saturated fatty acid. In other embodiments, the fatty acid substrate is a straight chain fatty acid, a branched chain fatty acid, or a fatty acid that includes a cyclic moiety.

Certain embodiments of the fatty acid conjugated peptoids include compositions having the structure of general formula IV or a pharmaceutically acceptable salt thereof.

| | Cn | Compound Name |
|---|---|---|
| 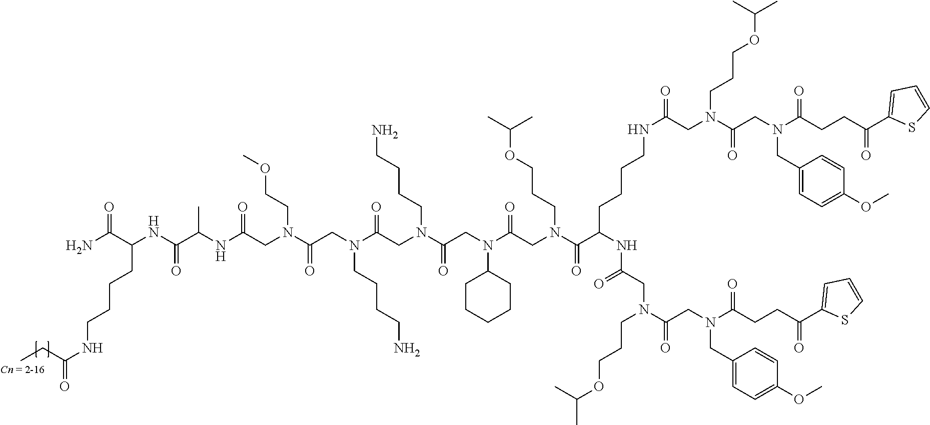 | 2 4 6 8 10 12 14 16 | PKR1-C4 PKR1-C6 PKR1-C8 PKR1-C10 PKR1-C12 PKR1-C14 PKR1-C16 PKR1-C18 |
| General Formula IV | | |

Certain embodiments of the fatty acid conjugated peptoids include compositions having the structure of general formula V, VI, or VII or a pharmaceutically acceptable salt thereof.

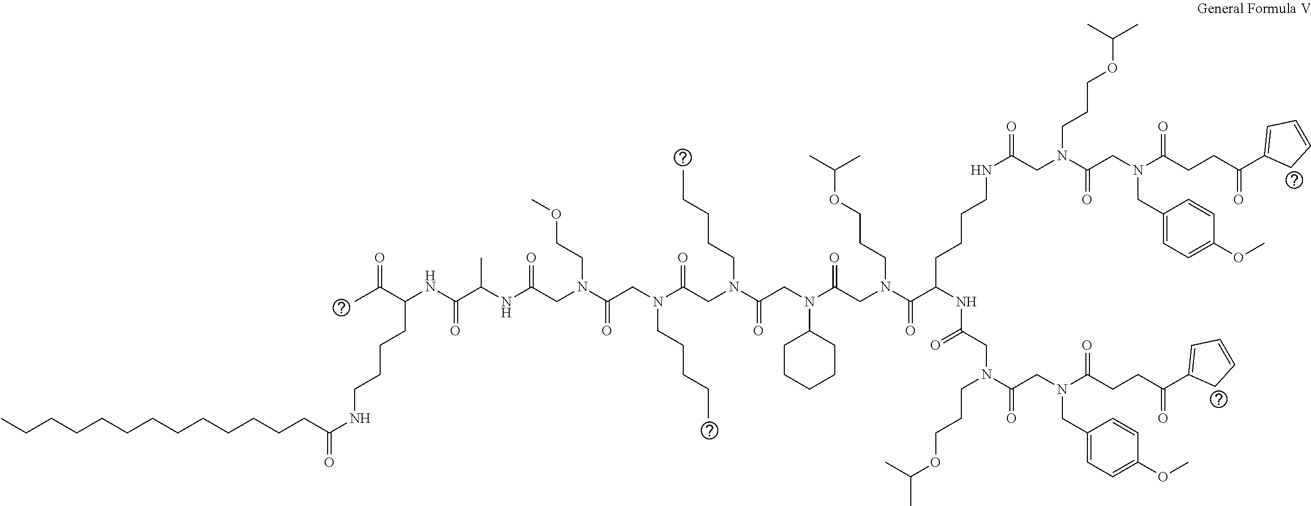

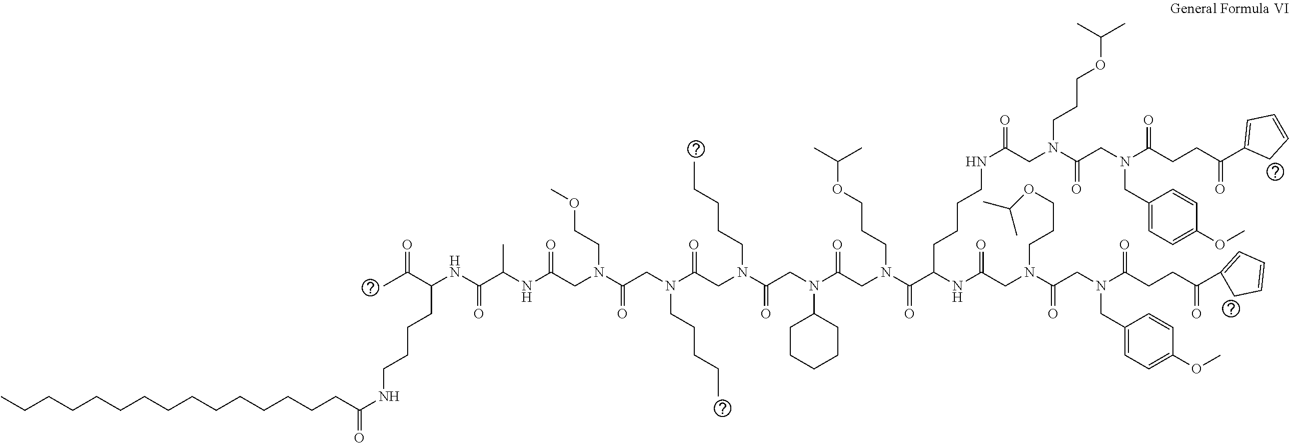

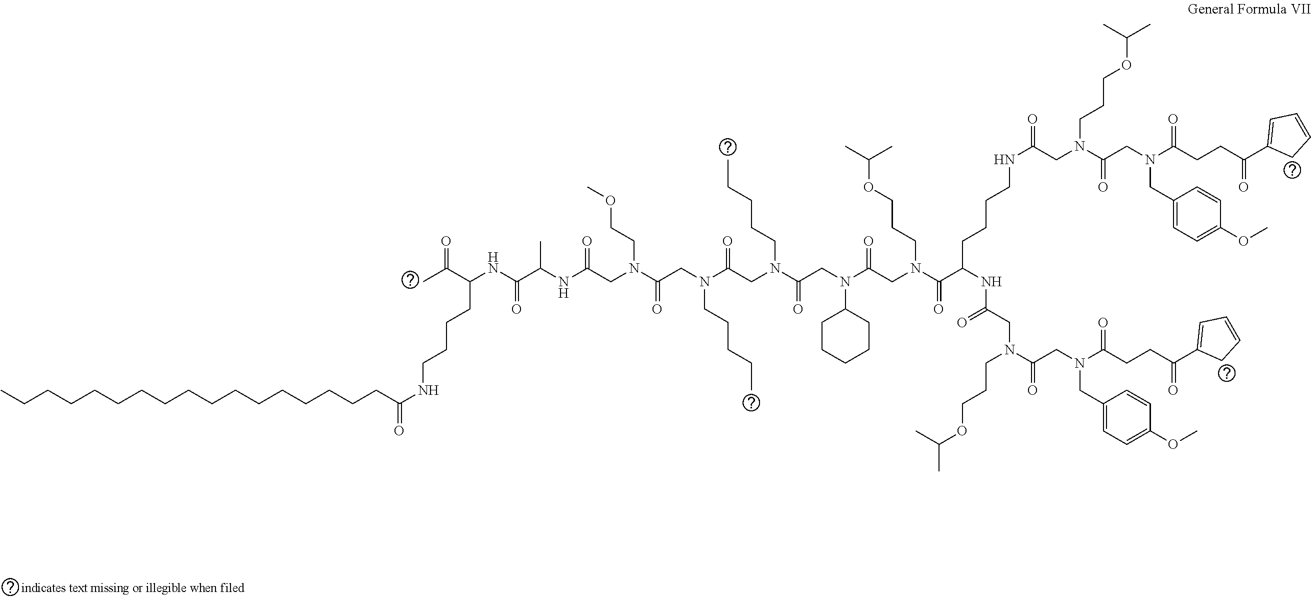

Embodiments include methods for treating cancer by administering to a subject suffering from or susceptible to cancer a peptoid inhibitor of a function of KRAS, HRAS, or NRAS. In certain embodiments, the present disclosure provides methods for treating cancer comprising administering to a subject suffering from or susceptible to cancer a peptoid inhibitor of mutant KRAS function. In certain embodiments, the KRAS is a mutant KRAS, which can be one of a KRASG12V, KRASG12C, KRASG12D, or KRASQ61H protein. In certain embodiments, the KRAS is a wild-type KRAS.

Peptoids refer to molecules designed to mimic functions of a peptide, but with improved serum stability, cell permeability and non-immunogenicity than peptides. Peptoids have their side chains on the amide nitrogen of the backbone, built by incorporating various synthetic organic groups derived from organic amines that do not occur in peptides built with natural amino acids. KRAS mutations can include single-base missense mutations at codon 12 (G12), codon 13 (G13), or codon 61 (Q61), such as G12C (glycine (GGT) to cysteine (TGT)), G12V, G12D, G12A, G12R, G13D, G13C, Q61H, or Q61R. KRASG12V is present in several cancers, with pancreatic adenocarcinoma, lung adenocarcinoma, colon adenocarcinoma, colorectal adenocarcinoma, and rectal adenocarcinoma having the greatest prevalence. In certain embodiments, the method further includes administering a MEK inhibitor or an ERK inhibitor to the subject suffering from cancer. The MEK inhibitor can be trametinib. The ERK inhibitor can be ulixertinib.

The binding affinity of the parent PKR1 for KRASG12V was examined by the quantitative ELISA-like binding assay and the Kd was found to be 573 nM, while the unbranched monomeric PKR1.M and the sequence scrambled KRA1.S lost this binding. The sarcosine scan was performed to identify minimum pharmacophore of PKR1, and found that residues at second, third, sixth, seventh, and eighth positions are important for the binding, whereas the residues at fourth position appears to hinder the binding. The anti-proliferative effects of PKR1 at cellular level were evaluated utilizing standard MTS assay with H441, H358, and H1993 cell lines, which harbor KRASG12V, KRASG12C, and KRASWT, respectively. To increase cell permeability and to anchor PKR1 onto the inner cell membrane closer to KRAS, fatty acids with different carbon chains were introduced at the C-terminal of PKR1. The MTS data indeed indicated that fatty acid conjugated compounds (PKR1-C14, PKR1-C16, and PKR1-C18) significantly improved the activity in H441 and H358 with IC50 ranging from 3.1μ M to 5.0μ.

The description may use the phrases “in certain embodiments,” “in various embodiments,” “in an embodiment,” or “in embodiments,” which may each refer to one or more of the same or different embodiments. Furthermore, the terms “comprising,” “including,” “having,” and the like, as used with respect to embodiments of the present disclosure, are synonymous.

A “pharmaceutical composition” refers to a mixture of one or more of the peptoids described herein, or a pharmaceutically acceptable salt, solvate, hydrate or prodrug thereof as an active ingredient, and at least one pharmaceutically acceptable carrier or excipient. The purpose of a pharmaceutical composition is to facilitate administration of a peptoid to a subject. In another aspect, the invention provides a pharmaceutical composition including a peptoid of one of the formulae described herein, or a pharmaceutically acceptable salt thereof, and a pharmaceutically acceptable carrier or excipient. In some embodiments, the pharmaceutical composition includes two or more pharmaceutically acceptable carriers and/or excipients.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use. The peptoids disclosed herein can exist in free form for treatment, or where appropriate, as a pharmaceutically acceptable salt. A “pharmaceutically acceptable salt” means any non-toxic salt of a peptoid that, upon administration to a recipient, is capable of providing, either directly or indirectly, a peptoid or an inhibitory active metabolite or residue thereof. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, glycolate, gluconate, glycolate, hemisulfate, heptanoate, hexanoate, hydrochloride, hydrobromide, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, palmoate, pectinate, persulfate, 3-phenylpropionate, phosphate, picrate, pivalate, propionate, salicylate, stearate, succinate, sulfate, tartrate, thiocyanate, p-toluenesulfonate, undecanoate, valerate salts, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Other acids and bases, while not in themselves pharmaceutically acceptable, may be employed in the preparation of salts useful as intermediates in obtaining the peptoids and their pharmaceutically acceptable acid or base addition salts.

“Therapeutically effective amount” means the amount of a compound that, when administered to a subject for treating a disease, is sufficient to effect such treatment for the disease. The “therapeutically effective amount” can vary depending on the compound, the disease and its severity, and the age, weight, etc., of the subject to be treated.

“Treating” or “treatment” of any disease or disorder refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both.

The quantity of peptoids administered will depend on the seriousness of the condition. For example, for treatment of an angiogenic condition, e.g., in the case of neoplastic tumor growth, the position and size of the tumor will affect the quantity of peptoids to be administered. In some embodiments, the peptoids can be administered via intradermal, intramuscular, intraperitoneal, intravenous, subcutaneous, oral, and/or gastric routes. The compositions herein are formulated in accordance to the mode of potential administration. Thus, if the composition is intended to be administered intranasally or by inhalation, for example, the composition may be a converted to a powder or aerosol form, as conventional in the art, for such purposes. Other formulations, such as for oral or parenteral delivery, are also used as conventional in the art. Compositions for administration herein may form solutions, suspensions, tablets, pills, capsules, sustained release formulations or powders.

Novel spider-like 3-dimensional (3D) peptoids can efficiently cover PPI surfaces of mutant KRAS. Certain embodiments include a 3D peptoid with high cell permeability. For example, an on-bead high throughput peptoid screening method targeting mutated KRASG12V was utilized to identify a peptoid, PKR1. The peptoid PKR1 binds to KRASG12V with a dissociation constant (Kd) of 573 nM, which is also the ligand concentration that binds to half the receptor sites at equilibrium. Certain embodiments of the peptoids include minimum pharmacophore of PKR1. Certain embodiments of the peptoids include fatty acid conjugated peptoids.

As the membrane bound KRAS proteins display different orientations in active states, short fatty acid chains were also added to PKR1 to anchor the peptoid to the membrane. The fatty acid conjugated peptoids, being membrane anchored 3D-peptoids, improved the activity over 25-fold, detected in the cellular cytotoxicity MTS assays.

### Identification of 3D-Peptoids Targeting KRAS

An on-bead high throughput screening (HTS) was performed to identify hit compounds targeting KRAS. Based on the understanding of the targeting site which keeps changing its shapes due to highly dynamic nature of the KRAS protein, more than 30,000 compound beads were utilized from the structurally diverse large 3D-peptoid library developed by the inventors. For the primary screening, the beads were incubated with biotinylated KRAS G12V and then subjected to treatment with streptavidin Qdots 655 (red).

FIG. 2A is a schematic representation of biotinylated 3D-peptoid bound his-tagged KRAS identification via streptavidin-Qdots 655. Two ‘hit’ beads were identified that emitted red color, which indicate that the 3D-peptoid displayed on that bead binds to KRAS G12V by the fluorescence microscope through the DAPI filter (FIG. 2B and FIG. 2C). FIG. 2B is a photograph of the isolated ‘hit 1’ bead after a primary screen and a secondary screen using procedure illustrated in FIG. 2A. FIG. 2C is a photograph of the isolated ‘hit 2’ bead after a primary screen and a secondary screen using procedure illustrated in FIG. 2A. Those two beads were then collected and processed to confirm that the binding was not non-specific conducting secondary screening which incubates the beads with only streptavidin Qdots 655. Absence of red color indicates that the prior red color was due to the binding of the 3D-peptoid on that bead to KRAS. Performing MS-MS sequencing of the two hit beads identified the structures of two hit compounds, PKR1 and PKR2. PKR1, which exhibited stronger red color, was selected for the further studies.

### Binding Affinity of PKR1 for KRAS G12V and WT KRAS

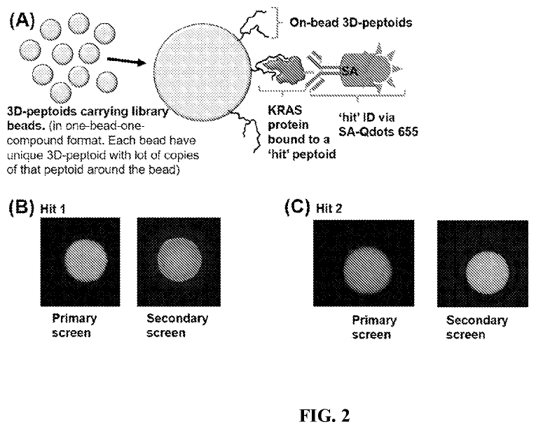

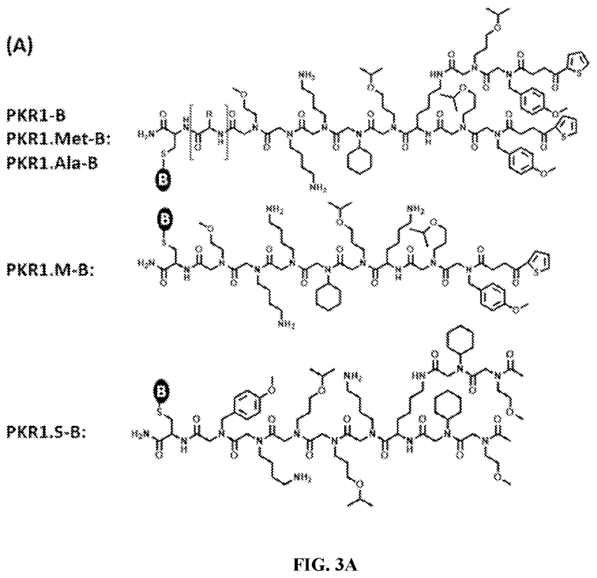

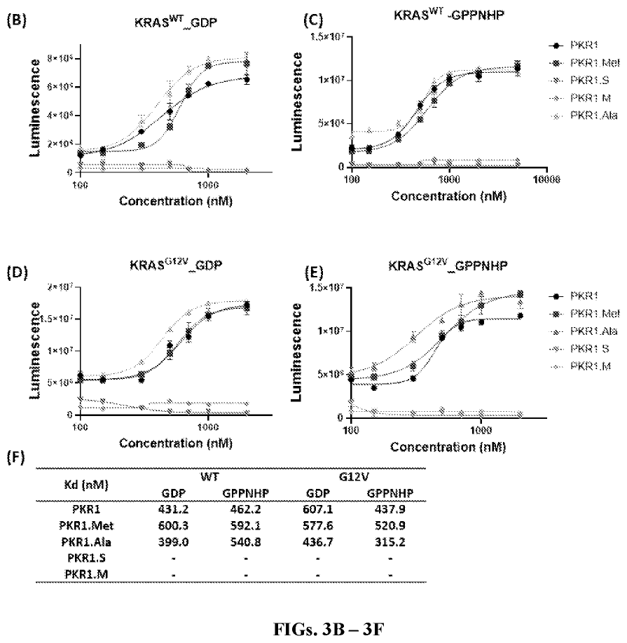

The binding affinity of the hit compound, PKR1, and its Ka value was evaluated by the quantitative ELISA-like binding assay. KRAS G12V and WT KRAS coated nickel coated 96 well plates were treated with a concentration gradient of biotinylated PKR1 derivatives in GDP/GPPNHP solutions and KRAS G12V and WT KRAS bound compounds were detected by streptavidin-horse radish peroxidase. FIG. 3A presents the chemical structure of biotinylated parent PKR1, PKR1.Met with Methionine, PKR1. Ala with Alanine, monomeric PKR1.M-B and scrambled PKR1.S-B utilized to ELISA-like binding assay. Compared to PKR1 scrambled compound, where the sequence of residues was rearranged (PKR1.S-B) and monomeric PKR1.M-B, which has no branched 3D structure, the parent hit compound PKR1 derivatives showed concentration-dependent binding. FIG. 3B and FIG. 3C are graphical representations of the concentration dependent WT KRAS binding curve of PKR1 derivatives, PKR1.M, and PKR1.S in GDP and GPPNHP solutions, respectively. FIG. 3D and FIG. 3E are graphical representations of the concentration dependent KRAS G12V binding curve of PKR1 derivatives, PKR1.M, and PKR1.S in GDP and GPPNHP solutions, respectively. FIG. 3F presents the Kd value of PKR1, PKR1.Met, PKR1.Ala, PKR1.S, and PKR1.M in WT KRAS and KRAS G12V with GDP and GPPNHP solutions. This result indicated that the branched 3D-peptoid can cover the large surface area of KRAS displaying an effective binding, while the linear peptoid with same sequence or sequence rearranged compound lost the binding. The comparable binding affinity observed for PKR1, PKR1.Met, and PKR1.Ala implies that methionine is a non-critical residue. PKR1 exhibits binding to KRAS in both active states, showing similar affinity, and binds to both KRAS G12V and WT KRAS with comparable binding affinity.

### Minimum Pharmacophore Identification of PKR1

To identify the residues most important for PKR1 compound to bind to KRAS, the minimum pharmacophore studies were performed by the replacement of each residue with sarcosine or acetyl group. At the treatment of sarcosine derivatives with 600 nM to KRAS G12V in the ELISA-like binding assay it was observed that the methyl group-replacement with residues at the second, third, sixth, seventh, and eighth positions decreased the binding to KRAS G12V. FIG. 4A is a graphical representation of the sarcosine derivatives binding results of PKR1 to KRASG12V. FIG. 4B provides a mapping of the results from the pharmacophore studies mapped onto the structure of PKR1. Especially, the substantial decrease of binding at seventh and eighth positions supported that p-methoxybenzyl amine at seventh position and 4-oxo-4-(thiophen-2-yl)butanone at eighth position play an important role in binding. Furthermore, cyclohexyl amine at fourth position is not only non-important for binding but also may be hindering the binding.

The MTS data indeed indicated that fatty acid conjugated compounds (PKR1-C14, PKR-C16, and PKR1-C18) significantly improved the activity in H441 lung cancer cells which contain KRASG12V (over 25-fold increase in potency). Those compounds also exhibited similar potency on KRASG12C expressed H358 lung adenocarcinoma cells. To optimize the length of fatty acids, more fatty acids with several different lengths (C4 to C18) can be utilized.

Biological Activity of PKR1 on Cancer Cell Lines with Different KRAS Mutations

The functional effects of PKR1 on cell survival and proliferation were evaluated utilizing the standard MTS cell viability assay. Various cancer cell lines with different KRAS mutations (WT, G12V, and G12C) were treated with PKR1 and PKR1.Met for 2 D (2 days). In these preliminary studies, PKR1 showed low activity with most cell lines having IC50 of >50 μM. FIGS. 5A-5F are graphical representations of the cell viability results of PKR1 and PKR1.Met in (A) H1993, (B) H1299, (C) H1693, (D) H441, (E) Capan2, (F) H358, and (G) H358 cell lines. This low activity of PKR1, which had very strong KRAS binding in nano-molar range, was suspected to be the result of cell permeability issues. Further improved derivatives were designed as described below. The data comparing PKR1 derivatives with Met (PKR1.Met) and Ala (PKR1) suggests that Met may be a non-important residue, thereby supporting the use of PKR1, which exhibits good yield, for further assays. The discrepancy between low activity at the cellular level and in vitro binding affinity assay indicated that 3D-branched peptoids compositions by themselves may face challenges related to cell permeability. H1299, H1693, and H1993 cells are non-small cell lung cancer (NSCLC) cell lines expressing KRAS WT. Capan2 cells are pancreatic cancer cell lines with a KRASG12V mutant. H2122 cells and H358 cells are lung cancer cell lines with a KRASG12C mutant.

Improvement of PKR1 by Adding Fatty Acid Component to Anchor PKR1 onto the Membrane Close to KRAS Proteins

The unmatched data from the in vitro binding assay and the cell viability assay indicates that the low activity under cellular level may be due to the low cell membrane permeability of PKR1 and also the low chance of an encounter with KRAS, which is anchored on the inner leaflet of cell membrane. Therefore, fatty acids were introduced at the C-terminal of PKR1 to improve the cell permeability as well as to facilitate the proper interaction with KRAS. Different lengths of fatty acids from butanoic acid (C4) to stearic acid (C18) were utilized to optimize the length of fatty acids. In other embodiments, the fatty acid is an unsaturated fatty acid, palmitoleic acid, or oleic acid.

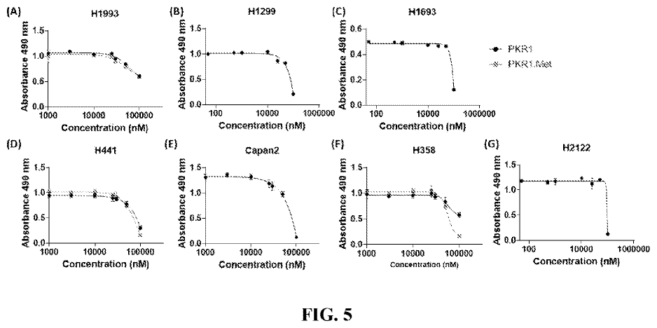

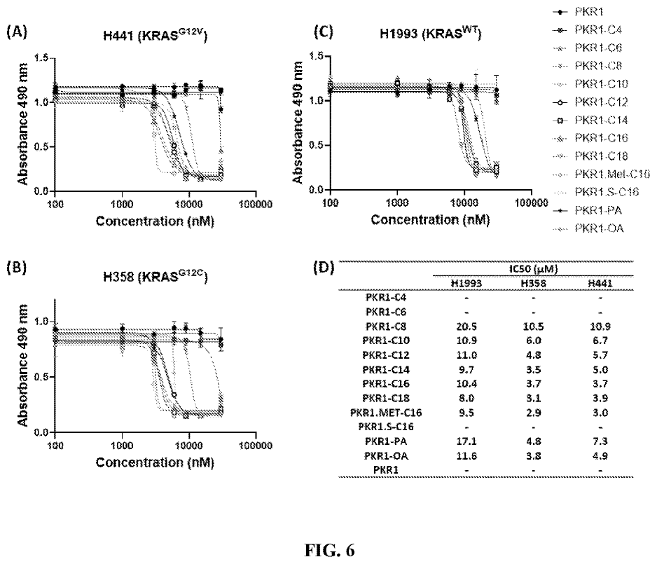

Three cancer cell lines (H441, H358, and H1993) harboring KRAS G12V, KRAS G12C, and WT KRAS, respectively, were subjected to treatment with the fatty acid conjugated-PKR1. The MTS data indicated that fatty acid conjugated compounds (from Cio to C18) significantly improved the activity in all cancer cell lines. FIGS. 6A-6C are graphical representations of the effects of the fatty acid conjugated PKR1 derivatives on cell survival and proliferation of (A) H441, (B) H358, and (C) H1993 cell lines. FIG. 6D presents the IC50 values of fatty acid conjugated PKR1 derivatives in H441, H1993, and H358 cell lines. PKR1-C16 showed significant improvement of activities in H441 and H358 more than in H1993 harboring WT KRAS. As KRAS is predominant in the membrane consisting of unsaturated fatty acids, conjugating unsaturated fatty acids to PKR1 can facilitate its anchoring onto the membrane, thereby improving efficacy. PKR1 derivatives conjugated with both saturated and unsaturated fatty acids exhibited similar effects on cell viability.

### Validation of Efficacy of Fatty Acid Conjugated PKR1 Derivatives in Cell Survival and Proliferation

Further assays utilizing PKR1-C16 were conducted to analyze the efficacy of PKR1 derivatives in cells. Timeline MTS assay was conducted to assess the effect of PKR1 derivatives on cell survival and proliferation. FIG. 7A is an illustration of the scheme of timeline MTS assay. After seeding cells overnight, 5 μM and 10 μM of PKR1-C16 and 10 UM of PKR1 as control were added. The efficacy by dosage was evaluated by daily treatment, one-time treatment, and two-time treatment. MTS was treated every 0.5 D, 1 D, 2 D, and 3 D. FIGS. 7B-7E are graphical representations of the cell viability following treatment with PKR1 and PKR1-C16 in H441 cell lines with (B) one time treatment, (C) two-time treatment at 0 D and 1 D, (D) two-time treatment at 0 D and 2 D, and (E) daily treatment. FIGS. 7F-7I are graphical representations of the cell viability following treatment with PKR1 and PKR1-C16 in H1993 cell lines with (F) one time treatment, (G) two-time treatment at 0 D and 1 D, (H) two-time treatment at 0 D and 2 D, and (I) daily treatment. Treatment with 5 μM of PKR1-C16 inhibited cell proliferation in the H441 cell lines, while a single exposure to the same concentration didn't impact proliferation in H1993 cell lines. Treatment with 10 μM of PKR1-C16 inhibited cell proliferation and survival in both H441 and H1993 cell lines. Every second treatment of PKR1-C16 inhibited cell proliferation and survival. These results indicate that for the compositions tested herein, the daily treatment resulted in the strongest inhibition compared to other dosing schedules tested herein.

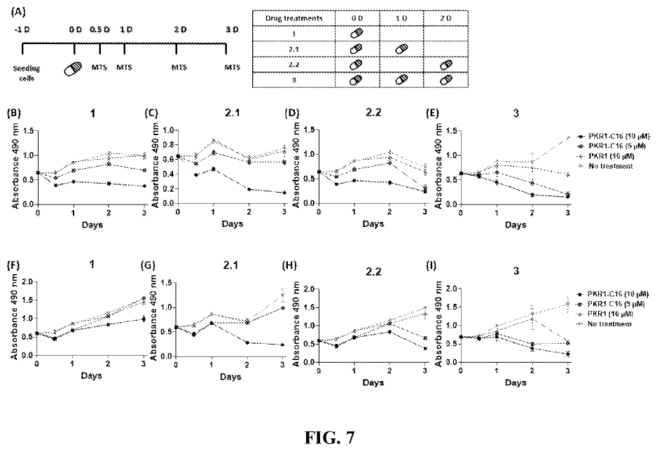

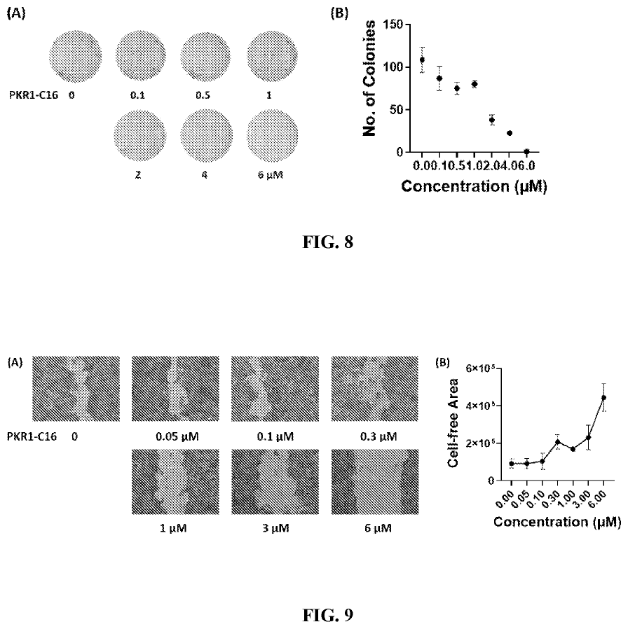

Colony formation assay was additionally performed with PKR1-C16 in a concentration gradient in H441 cell lines. FIG. 8A is a set of representative images of colonies for H441 after 9 D incubation with a concentration gradient of PKR1-C16. Black dots represent colonies. Colonies were stained with 20% crystal blue solution after 9 D incubation with PKR1-C16 and colonies were counted utilizing the Fiji program. FIG. 8B is a graphical representation presenting the dose-response relationship of number of colonies in H441 cells. PKR1-C16 exhibited a dose-dependent inhibition of colony formation in H441 cell line, indicating that its effect on cell proliferation.

### Validation of Efficacy of Fatty Acid Conjugated PKR1 Derivatives in Cell Migration

Wound scratch assay, which is a common technique to study in vitro cell migration, was performed using H441 cell lines. FIG. 9A is a set of representative images of wound healing for H441 cell lines after 2 D incubation with a concentration gradient of PKR1-C16. Magnification 20×. Cells were stained after 2D incubation with a concentration gradient of PKR1-C16. Cell-free area was determined via Fiji Wound Healing Tool. FIG. 9B is a graphical representation presenting the dose-response relationship of cell-free area in H441 cells. Treatment with PKR1-C16 at concentrations above 300 nM resulted in the inhibition of cell migration.

### PKR1 Derivatives as Pan-KRAS Inhibitors

To examine the capacity of PKR1 derivatives as broad-spectrum KRAS inhibitors, MTS assays were conducted in various types of cell lines representing different cancer types, KRAS mutations, and KRAS gene zygosity (Table 1). IC50 values for each cell line were obtained from the MTS assay with PKR1-C16 treatment.

TABLE 1
Lung non-cancer and cancer cell lines by KRAS
mutation, amplification, and zygosity.

| | | | KRAS | | IC₅₀ |
|---|---|---|---|---|---|
| NAME | TISSUE | Cancer | MUTATION | AMPLIFICATION | (μM) |
| HBEC3-KT | Lung | x | WT | — | 40.3 |
| H1693 | Lung | yes | WT | — | >15 |
| H1993 | Lung | yes | WT | — | 10.4 |
| H1299 | Lung | yes | WT | — | >15 |
| H1395 | Lung | yes | WT | — | 7.6 |
| H441 | Lung | yes | G12V (HETERO) | yes | 3.7 |
| H358 | Lung | yes | G12C (HETERO) | yes | 3.7 |
| H2122 | Lung | yes | G12C (HOMO) | no | 6.1 |
| H1155 | Lung | yes | Q61H (HOMO) | — | 4.6 |

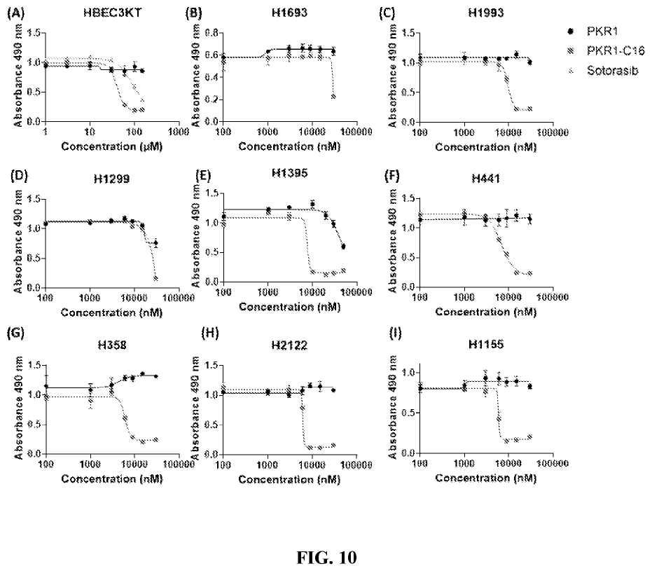

PKR1-C16 exhibited greater activity, with an IC50 of approximately 5 μM, in the KRAS mutant cell line compared to the WT KRAS cell line, with an IC50 of around 10 μM. FIGS. 10A-10I are graphical representations of the cell viability results of PKR1 derivatives in (A) HBECK3KT, (B) H1693, (C) H1993, (D) H1299, (E) H1395, (F) H441, (G) H358, (H) H2122, and (I) H1155 cell lines. PKR1-C16 exhibited a decrease of activity in non-cancer cells, approximately 10 times less potent than in KRAS mutant cancer cell lines. Despite the parent PKR1 binding to both WT KRAS and KRAS mutant with similar affinity, PKR1 derivatives exhibited greater inhibition in KRAS mutant cancer cells compared to WT KRAS cancer cells or non-cancer cells (i.e. normal cells). The observed difference in inhibition effectiveness may be attributed to higher KRAS expression levels in mutant cells compared to WT cells.

### Sensitization of Heterozygous KRAS Mutant Cancer Cells for Downstream Signaling Inhibitors

Recent studies indicate that homozygous KRAS mutant cell lines lacking WT KRAS allele exhibit greater sensitivity to MEK or ERK inhibitors compared to heterozygous KRAS mutant cells with presence of one copy of WT KRAS allele. Trametinib and Ulixertinib, functioning as MEK and EKR inhibitors respectively, exhibit higher potency in H2122 cells carrying homozygous KRAS G12C mutations compared to H23 cells with heterozygous KRAS G12C mutations.

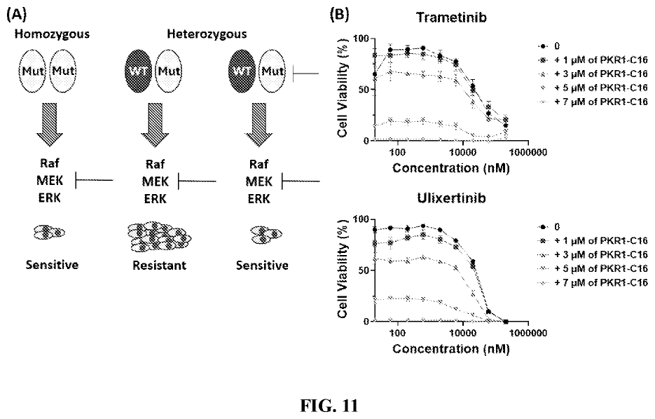

Provided herein are pan-KRAS inhibitors that can enhance the sensitivity of heterozygous KRAS mutant cells to KRAS downstream inhibitors, thereby overcoming limitations in their use for cancer therapy. FIG. 11A is an illustration presenting that heterozygous KRAS mutant cells are resistant to the inhibition of KRAS downstream signaling while homozygous KRAS mutant cells are sensitive to such inhibition, therefore blocking wild type KRAS potentially re-sensitizes the heterozygous KRAS mutant cells to the inhibition of KRAS downstream signaling. Cell viability of H441 cells harboring heterozygous KRAS G12V were assessed in combination treatment with PKR1-C16. FIG. 11B is a graphical representation presenting the cell viability of H441 harboring heterozygous KRAS G12V with co-treatments of Trametinib (upper panel) and Ulixertinib with PKR1-C16 (lower panel). The single treatment of Trametinib and Ulixertinib for H441 cells during a 3 D incubation resulted in IC50 values of 10.4 μM and 17.0 μM, respectively. Concentration gradients of Trametinib and Ulixertinib were co-treated with 1, 3, 5, and 7 μM of PKR1-C16, respectively. Co-treatments with PKR1-C16 resulted in a dose responsive inhibition of cell viability. Especially, co-treatment with PKR1-C16 above a concentration of 3 μM significantly inhibited cell viability. Blocking WT KRAS enables the re-sensitization of heterozygous KRAS mutant cells to the inhibitors of KRAS downstream signaling.

### Membrane Localization of Fatty Acid Conjugated PKR1 Derivatives

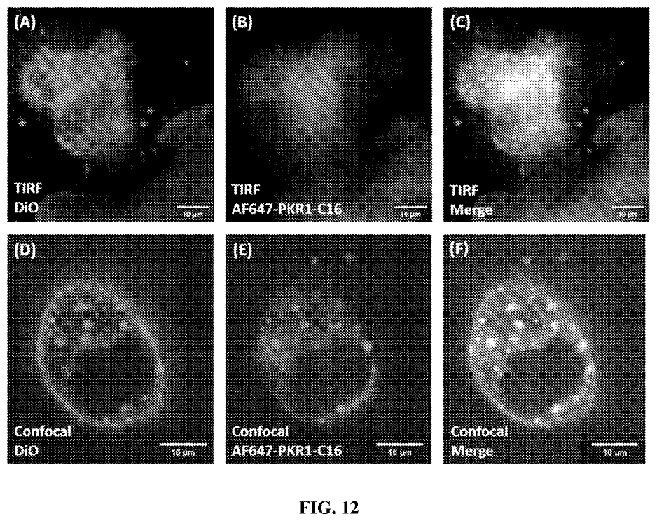

To identify whether fatty acid conjugated PKR1 localizes to the inner membrane, Total Internal Reflection Fluorescence (TIRF) was performed to visualize fluorescently labeled molecules near the plasma membrane with high spatial resolution, and confocal microscopy was performed to acquire 3D images of the cell membrane. H441 cells on a u-Slide 8 Well plate were treated with AF647 labeled PKR1-C16 (excitation: 650 nm, emission: 671 nm) and DiO (excitation: 483 nm, emission: 501 nm), a membrane dye. Cells were imaged on Nikon Eclipse Ti2 microscope. TIRF and confocal images were obtained utilizing 100× and 60 × oil objective, respectively. FIGS. 12A-12F are TIRF microscopy images of (A) DiO, (B) AF647-PKR1-C16, and (C) merge of DiO and AF647-PKR1-C16, and confocal microscopy images of (D) DiO, (E) AF647-PKR1-C16, and (F) merge of DiO and AF647-PKR1-C16. In FIGS. 12A-12C, AF647-PKR1-C16 exhibited a membrane localization visualized with DiO, indicating its potential interaction or targeting of membrane constituents. Additionally, confocal microscopy images demonstrated intracellular localization of PKR1-C16 within the cytoplasm (FIGS. 12D-12F). Fatty acid conjugation facilitates the cell permeability and may anchor the PKR1-C16 molecules onto the membrane.

### Molecular Docking Study of PKR1 and Fatty Acid Conjugated PKR1 on Membrane-Bound KRAS

The conventional KRAS inhibitors have been developed by the structure-based drug design with only KRAS crystal structures. However, as KRAS is anchored to the inner leaflet of cell membrane, KRAS structures were analyzed under cellular conditions. KRAS activation state determines population of two major orientations on lipid bilayer. In the major cluster for the GDP-bound form, most of KRAS helices are oriented parallel to the membrane and the dimeric interface of KRAS comprised of α4, β6, and α5 is occluded by the lipid membrane. Moreover, GTPase domain containing Switch I and II is located opposite to the dimeric interface and exposed. However, the helices are oriented semi-perpendicular to the membrane in the cluster that predominates in activated KRAS GMPPNP, an analog of GTP. A2 and β1-3 of KRAS form the membrane bound interface and the dimeric interface is exposed, supporting the study that activated KRAS form homodimer to recruit its downstream signaling proteins such as Raf. GTPase domain is partially occluded, requiring the reorientation toward a fully exposed orientation by the interaction with the downs streaming signaling proteins such as Raf-RBD. Due to these highly dynamic orientations of KRAS, the optimizations of peptoid drug-leads and the linker between fatty acids and peptoid were conducted with the consideration of membrane bound structures of KRAS. Certain embodiments of the fatty acid conjugated peptoid include a linker. In certain embodiments, the linker can be one or more amino acids or derivatives thereof. In certain embodiments, the linker is up to five amino acids long. In certain embodiments, the fatty acid is coupled with s-amino group of lysine at the C-terminal of the peptoid. Therefore the linker in this embodiment is one lysine residue.

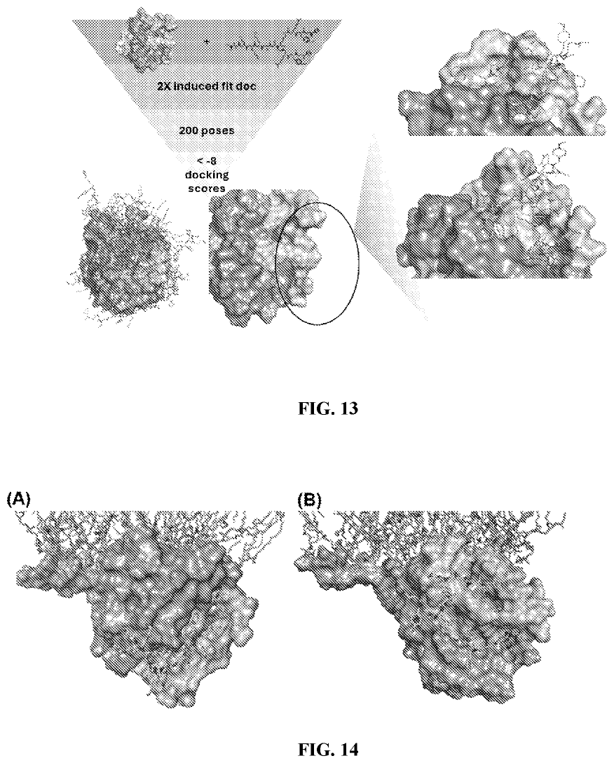

Before performing induced-fit docking in membrane bound KRAS, a promising binding mode of PKR1 to mono KRAS protein was identified. Induced-fit docking of PKR1 for GDP bound KRAS (PDB: 7C40) and GPPNHP bound KRAS (PDB: 6GOE) were performed utilizing molecular operating environments (MOE) program, repeating the process twice respectively. FIG. 13 presents a schematic strategy to identify the most potential binding mode of PKR1 derivatives on KRAS. About 200 poses were obtained during each run and poses with docking scores below-8 were selected for further analysis. The results were clustered to identify the most potential binding sites, revealing that many poses are found in the GTPase domain or dimer interface. Among them, the binding poses compatible with minimum pharmacophore scanning were identified, indicating that it can inhibit the access of KRAS upstream and downstream signaling proteins.

Next, induced-fit molecular docking of PKR1 for GDP bound KRAS (PDB: 2MSC) and GMPPNP bound KRAS (PDB: 2MSD) was performed. FIGS. 14A and 14B present molecular docking poses of PKR1 to (A) GDP bound KRAS and (B) GMPPNP bound KRAS. Similar to the results from the docking study with mono KRAS, PKR1 was fittingly spread out to the large surface area including GTPase site on both GDP and GMPPNHP bound KRAS. Interestingly, the C-terminal of PKR1 is close to the membrane in the docking pose from GMPPNP bound KRAS.

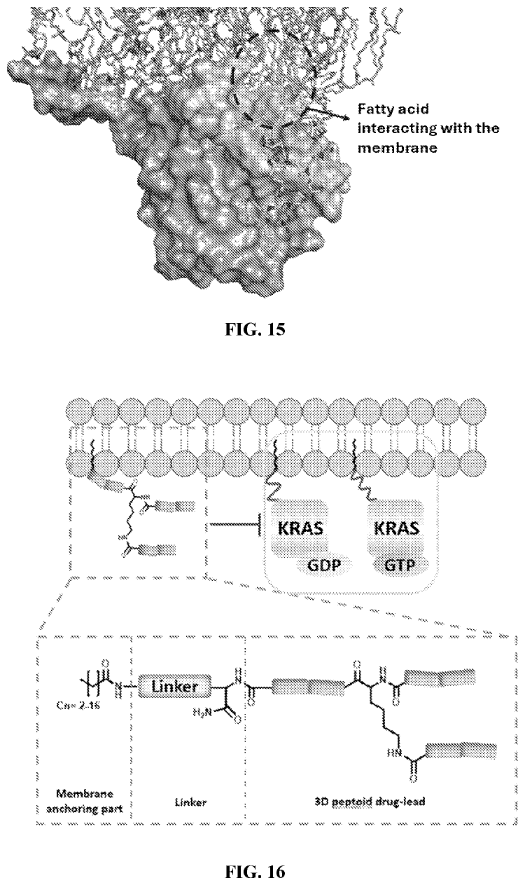

The docking study of myristic acid conjugated PKR1 (PKR1-C14) was conducted. FIG. 15 presents the molecular docking results of PKR1-C14 to GMPPNP bound KRAS. In this preliminary docking study, the peptoid drug-lead portion of PKR1-C14 appeared to be closer to GTPase area (still bound within the GTPase domain) allowing the myristic acid portion to interact with the membrane bound interface. In certain embodiments, introduction of the linker between the fatty acid and the peptoid drug-lead may position the compound to precisely recognize their intended binding hotspots (i.e. fatty acid is anchored onto the inner lipid membrane and peptoid drug-lead landing on the binding sites on GTPase domain, where the parent compound, PKR1, was placed). FIG. 16 presents the overview of the mechanism of 3D-peptoid for KRAS signaling and schematic representation of membrane anchored 3D-peptoid structure.

The branched 3D-peptoids perform as pan-KRAS inhibitors, with the potential to serve as widely applicable novel cancer therapeutics. Fatty acid conjugation enhances the cellular activities of 3D-peptoids, such as PKR1. These results can be attributed to the enhanced cell permeability and membrane localization through fatty acid conjugation. Fatty acid conjugated PKR1-C16 significantly inhibited KRAS mutant cell lines more effectively than WT KRAS cell lines. PKR1-C16 exerts antagonistic effects on cell proliferation, survival, and migration. Combination treatments of PKR1-C16 with KRAS downstream signaling inhibitors sensitized heterozygous KRAS mutant cancer cells.

Certain embodiments include multimerized PKR1, enabling a single molecule to target multiple proteins simultaneously. These compositions can significantly enhance the potency of PKR1. Certain embodiments of the modified PKR1 derivatives are conjugated with palmitic acid.

Other objects, features and advantages of the disclosure will become apparent from the foregoing drawings, detailed description, and examples. These drawings, detailed description, and examples, while indicating specific embodiments of the disclosure, are given by way of illustration only and are not meant to be limiting. In further embodiments, features from specific embodiments may be combined with features from other embodiments. For example, features from one embodiment may be combined with features from any of the other embodiments. In further embodiments, additional features may be added to the specific embodiments described herein. It should be understood that although the disclosure contains certain aspects, embodiments, and optional features, modification, improvement, or variation of such aspects, embodiments, and optional features can be resorted to by those skilled in the art, and that such modification, improvement, or variation is considered to be within the scope of this disclosure.

What is claimed is:

1. A peptoid having a structure of general formula I or II or III or a pharmaceutically acceptable salt thereof.

2. The peptoid of claim 1, further comprising a C4-C18 fatty acid at a C-terminal of the structure of general formula I or II or III or a pharmaceutically acceptable salt thereof.

3. The peptoid of claim 2, wherein the fatty acid is myristic acid, palmitic acid, or stearic acid.

4. A method for treating cancer, the method comprising:
administering to a subject suffering from or susceptible to cancer a pharmaceutical composition containing a peptoid of claim 1.

5. The method of claim 4, wherein the peptoid inhibits function of a KRAS protein in a cancer cell.

6. The method of claim 5, wherein the KRAS protein is a mutant KRAS protein or a wild type KRAS protein.

7. The method of claim 5, wherein the cancer cell overexpresses wild type KRAS as compared to a normal cell.

8. The method of claim 6, wherein the mutant KRAS is a KRASG12V, KRASG12C, KRASG12D, or KRASQ61H protein.

9. The method of claim 4, wherein the pharmaceutical composition includes a C4-C18 fatty acid conjugated at a C-terminal of the peptoid.

10. The method of claim 9, wherein the fatty acid is myristic acid, palmitic acid, or stearic acid.

11. The method of claim 9, wherein the fatty acid is an unsaturated fatty acid, palmitoleic acid, or oleic acid.

12. The method of claim 4, wherein the cancer is an adenocarcinoma.

13. The method of claim 4, further comprising:
administering a therapeutically effective amount of a MEK inhibitor or an ERK inhibitor to the subject suffering from cancer.

14. The method of claim 13, wherein the MEK inhibitor is trametinib.

15. The method of claim 13, wherein the ERK inhibitor is ulixertinib.
